(19) United States
(12) Patent Application Publication
(10) Pub. No.: US 2026/0014033 A1
(43) Pub. Date: Jan. 15, 2026

(54) ABSORBENT ARTICLE

(71) Applicant: THE PROCTER & GAMBLE COMPANY, Cincinnati, OH (US)

(72) Inventors: Sijia WANG, Beijing (CN); Runze QIN, Beijing (CN); Wanning LI, Beijing (CN)

(21) Appl. No.: 19/258,990

(22) Filed: Jul. 3, 2025

(30) Foreign Application Priority Data
Jul. 9, 2024 (WO) PCT/CN2024/104403
Aug. 7, 2024 (WO) PCT/CN2024/110290

(51) Int. Cl. A61F 13/47; A61F 13/15

(52) U.S. Cl. CPC A61F 13/47; A61F 13/15203; A61F 2013/15463

(57) ABSTRACT

The present disclosure relates to an absorbent article comprising a liquid-permeable topsheet, a backsheet, an absorbent core disposed between the topsheet and the backsheet, and a patch disposed between the topsheet and the absorbent core, wherein the patch has a width in the range of about 30% to about 80% of a width of the absorbent core, and a length in the range of about 30% to about 75% of a length of the absorbent core, wherein the patch has an acquisition time no higher than about 5 seconds as measured according to Material Acquisition Time Test, and wherein the patch has a xy-direction distribution of at least about 0.8 cm² as measured according to XY-direction Distribution Test.

### CROSS REFERENCE TO RELATED APPLICATION

This application claims priority to PCT Patent Application Serial Nos. PCT/CN2024/104403 filed Jul. 9, 2024 and PCT/CN2024/110290 filed Aug. 7, 2024, the entire disclosures of all which are fully incorporated by reference herein.

### FIELD OF THE INVENTION

The present invention relates to absorbent articles comprising a liquid permeable topsheet, a liquid impermeable backsheet, an absorbent core disposed between the topsheet and the backsheet, and a patch disposed between the topsheet and the absorbent core.

### BACKGROUND OF THE INVENTION

Absorbent articles such as diapers and sanitary napkins are typically composed of multiple components such as a liquid-permeable topsheet, liquid-impermeable backsheet and a liquid-storing absorbent core disposed the topsheet and the backsheet, and it is desirable that body fluid on the topsheet is effectively drawn from the topsheet and promptly moves to components underneath the topsheet such as an absorbent core without remaining in the topsheet.

Users of such disposable absorbent articles have several concerns including leakage from products like catamenial pads, diapers, sanitary napkins, and incontinence pads. To prevent fluid leakage, absorbent articles are desired to provide a fast acquisition speed.

Absorbent articles commonly employ a fluid management layer between a topsheet and an absorbent core which can quickly receive large amounts of fluid from a topsheet and temporarily store it before the fluid is absorbed by the absorbent core. Therefore, one desirable function of a fluid management layer is to quickly acquire fluid from a topsheet and transfer it to the absorbent core in an efficient manner.

WO2014/101927A discloses an absorbent article comprising a fluid flow control member which is a spacer woven fabric located between a first absorbent layer and a second absorbent layer, the spacer woven fabric comprising a top layer, a bottom layer and an interconnecting layer of yarns between the top layer and the bottom layer.

Meanwhile, through the use of innovative materials as components such as a topsheet, a fluid management layer, and absorbent gelling materials, absorbent articles have drastically advanced to provide women with products that absorb menses and transfer fluids away from a woman's body. However, given complexity of material properties affecting fluid acquisition performance, it is not easy to design an absorbent product to utilize the product's overall acquisition capacity.

Based on the foregoing, there is a need for absorbent articles designed to effectively utilize absorbent capacities of components of the absorbent article.

### SUMMARY

The present invention provides an absorbent article comprising a liquid-permeable topsheet, a backsheet, an absorbent core disposed between the topsheet and the backsheet, and a patch disposed between the topsheet and the absorbent core, wherein the patch has a width in the range of about 30% to about 80% of a width of the absorbent core, and a length in the range of about 30% to about 75% of a length of the absorbent core, wherein the patch has an acquisition time no higher than about 5 seconds as measured according to Material Acquisition Time Test, and wherein the patch has a xy-direction distribution of at least about 0.8cm² as measured according to XY-direction Distribution Test.

These and other features, aspects, and advantages of the present invention will become evident to those skilled in the art from a reading of the present disclosure.

### BRIEF DESCRIPTION OF THE DRAWINGS

FIG. 1 is a perspective view of an embodiment of an absorbent article according to the present invention.

FIG. 2 is a lateral cross-section view along 2-2 of the absorbent article of FIG. 1.

FIG. 3 is a schematic drawing illustrating measurement of sizes of an absorbent core and a patch.

FIGS. 4A-4F are microscopic images from the topsheet side of sanitary napkins with patch material 1 in different patch sizes in Stain Size Test.

FIGS. 5A-5F are microscopic images from the topsheet side of sanitary napkins with patch material 2 in different patch sizes in Stain Size Test.

FIG. 6A is a perspective view of a strikethrough plate for acquisition time measurement.

FIG. 6B is a plan view of the strikethrough plate of FIG. 6A.

FIG. 6C is a plan view of a 6C-6C direction cross section of the strikethrough plate of FIG. 6B.

FIG. 6D is a plan view of part of the strikethrough plate of FIG. 6B.

FIG. 6E is a plan view of a 6E-6E direction cross section of the strikethrough plate of

FIG. 6B.

### DETAILED DESCRIPTION

All ranges are inclusive and combinable. The number of significant digits conveys neither limitations on the indicated amounts nor on the accuracy of the measurements. All numerical amounts are understood to be modified by the word “about” unless otherwise specifically indicated.

The term “absorbent articles”, as used herein, include disposable diapers, sanitary napkins, panty liners, incontinence pads, interlabial pads, breast-milk pads, sweat sheets, animal-use excreta handling articles, animal-use diapers, and the like.

The term “absorbent core”, as used herein, refers to the component of the absorbent article that is primarily responsible for storing liquids. As such, the absorbent core typically does not include the topsheet or backsheet of the absorbent article.

The term “cellulose-based fibers”, as used herein, intends to include both cellulose fibers such as pulp and cotton, and regenerated cellulose fiber such as rayon unless specified differently.

The term “component” of an absorbent article, as used herein, refers to an individual constituent of an absorbent article, such as a topsheet, acquisition layer, liquid handling layer, absorbent core or layers of absorbent cores, backsheets, and barriers such as barrier layers and barrier cuffs.

The term “joined”, as used herein, refers to the condition where a first component is attached, or connected, to a second component either directly or indirectly. Where the first component is attached, or connected, to an intermediate component which in turn is attached, or connected, to the second component, the first component and second component are joined indirectly.

As used herein, the term “layer” refers to a three dimensional structure having two dimensions that are substantially greater than the third dimension. The term layer is not limited to single layers or sheets of material. Thus a layer may comprise laminates or combinations of several sheets or webs of the requisite type of materials. Accordingly, the term “layer” includes the terms “layers” and “layered.”

### Absorbent Article

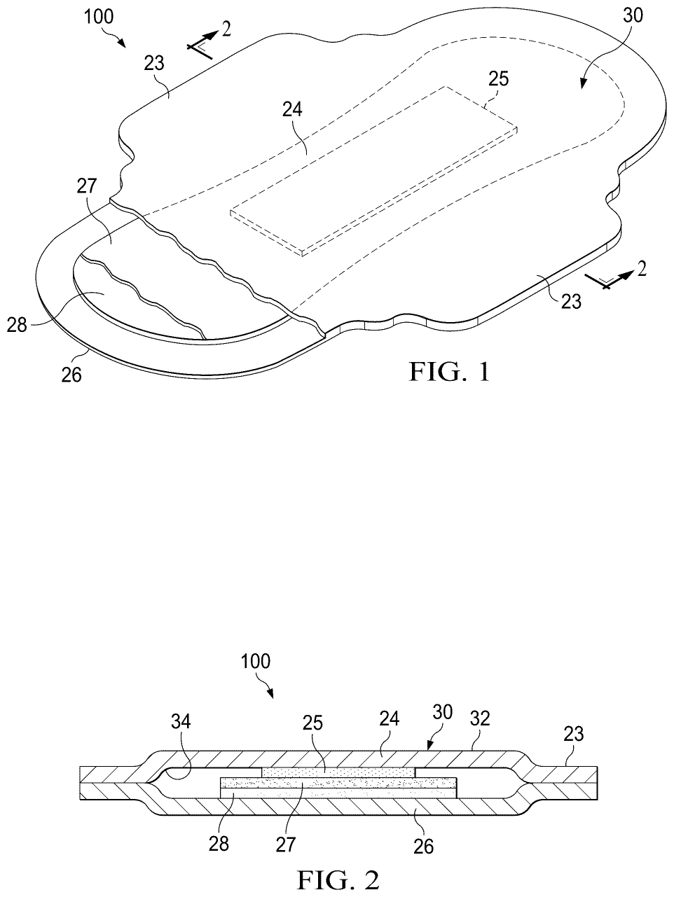

Absorbent articles will now be generally discussed and further illustrated in the form of a sanitary napkin 100 as exemplarily represented in FIGS. 1 and 2. FIG. 1 is a plan view of the exemplary sanitary napkin 100 in a flattened-out configuration and the wearer facing side visible. FIG. 2 is a lateral cross-section view along 2-2 of the absorbent article of FIG. 1.

Referring to FIGS. 1 and 2, an absorbent article according to the present disclosure, a sanitary napkin 100 for example, comprises a topsheet 24 having a wearer facing surface and a garment facing surface positioned opposite to the wearer facing surface. The topsheet 24 comprises a top surface 32 forms the wearer facing surface of the absorbent article.

The absorbent article further comprises a backsheet 26 having a garment facing surface and a wearer facing surface positioned oppositely to the garment facing surface. The topsheet and the backsheet are preferentially peripherally joined using known techniques, either entirely so that the entire perimeter of the absorbent article is circumscribed by such joinder or are partially peripherally joined at the perimeter.

The absorbent article also comprises an absorbent core 28 positioned between the topsheet 24 and the backsheet 26. The absorbent article comprises a patch layer 25 positioned between the topsheet 24 and the absorbent core 28.

The absorbent article may further comprise additional fluid management layer 27 (or system). The absorbent article may further comprise a pair of flaps or wings 23. The topsheet 24, the backsheet 26, the patch layer 25, and the absorbent core 28, and other optional elements can be assembled in a variety of well-known configurations.

When the absorbent article is a sanitary napkin as shown in FIG. 1, as it is typical for sanitary napkins and the like, the sanitary napkin can have panty-fastening adhesive disposed on the garment facing side of backsheet 26. The panty-fastening adhesive can be any of known adhesives used in the art for this purpose, and can be covered prior to use by a release paper, as is well known in the art. If flaps or wings are present, panty fastening adhesive can be applied to the garment facing side so as to contact and adhere to the underside of the user's panties.

### Topsheet

The absorbent article comprises topsheet 24. The topsheet is a layer of the article that contacts the body of the wearer and receives bodily discharges. The topsheet is liquid pervious and may be flexible and non-irritating to the skin. The term “liquid pervious” as used herein refers to components that allow liquids to pass therethrough without significantly retarding or obstructing the transmission of such liquids therethrough. The topsheet may be a nonwoven layer, a polymeric film layer or a laminate comprising a nonwoven layer or a polymeric film layer.

Nonwoven suitable for the topsheet may comprise thermoplastic fibers. Thermoplastic fibers may be selected from the group consisting of polyesters, polypropylenes, polyethylenes, polyethers, polyamides, polyhydroxyalkanoates, polysaccharides, and combinations thereof. Additionally, other synthetic fibers such as rayon, polyethylene, and polypropylene fibers can be used within the scope of the present disclosure. Thermoplastic fibers may be single component fibers (i.e., single synthetic material or a mixture to make up the entire fiber), multicomponent fibers such as bicomponent fibers or combinations thereof.

The nonwoven may comprise cellulose-based fibers. Several examples of the nonwoven may include, but are not limited to: spunbonded nonwovens; carded nonwovens; carded air through nonwovens; spunlace nonwovens, needle punched nonwovens and nonwovens with relatively specific properties to be able to be readily deformed.

The topsheet may comprise a plurality of discrete features. Suitable configurations for the features include, but are not limited to, apertures; ridges (continuous protrusions), discontinuous protrusions, and grooves (continuous depressions), recesses; and any combination thereof. Such discrete features may be formed according to a known process.

The nonwoven constituting the topsheet in the present disclosure may have a basis weight from about 20 to about 100 g/m², or from about 30 to about 60 g/m², or about 20 to about 50 g/m^{2,}or about 30 to about 50 g/m².

### Backsheet

The absorbent article comprises backsheet 26. The backsheet may be any flexible, liquid resistant, and liquid impervious material. The backsheet prevents discharges collected by and contained in the sanitary napkin, and particularly discharges absorbed by the core, from escaping the sanitary napkin and soiling the clothing and bedding of the wearer. Any conventional backsheet materials may be used within the invention, such as polyolefinic films.

The backsheet typically extends across the whole of the absorbent structure and can extend into and form part of or all of side flaps, side wrapping elements or wings, when present.

### Absorbent Core

The absorbent article of the invention comprises an absorbent core 28 disposed between the topsheet 24 and the backsheet 26.

The absorbent core comprises an absorbent material. The absorbent material in the absorbent core can be any liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it did not exclude that other forms of SAP may be used such as a superabsorbent polymer foam, for example.

The size and shape of the absorbent core can be altered to meet absorbent capacity requirements, and to provide comfort to the wearer. As with the other elements of the articles of the invention, there are no particular requirements for the absorbent core and any standard absorbent material known in the art for use in absorbent articles will normally be suitable.

### Patch

The absorbent article comprises a patch disposed between the topsheet and the absorbent core. To rapidly and effectively transfer fluid the body discharged on the top surface of an absorbent article towards the absorbent core, an absorbent article comprises a fluid management layer between the topsheet and the absorbent core. To achieve the purposes, it is desirable that the fluid management layer has not only a fast vertical acquisition speed in the thickness of the fluid management layer for instant fluid gush handling, but also rapid distribution along the length and the width directions of the absorbent product so that the fluid absorption capacity of the underlying storage layer such as an absorbent core can be fully utilized in its entire area. However, when the xy-direction distribution of the flow is too fast, it results in a relatively wide colored region perceived from the top side of the absorbent article even with a small fluid amount. It is believed that perceptions of absorbency may be affected by visual impressions, i.e., its visual appearance, and if an absorbent article product during usage is seen with a colored area reaching close to any edges in the product, the consumer tends to think that the product uses up its absorbent capacity and concerns an immediate leakage risk untimely. Therefore, it is critical to leverage a superior fast acquisition speed and a strong xy-direction distribution of the fluid management layer without causing a negative perception on protection and compromising on leakage as well.

The patch of the present disclosure has an excellent z-direction distribution speed, and comprises an acquisition time no higher than about 5 seconds, or no higher than about 4 seconds, or no higher than about 3.5 seconds as measured according to Material Acquisition Time Test. Further, the patch in the present invention has an excellent xy-plane direction distribution speed, and comprises a distribution capacity of at least about 0.8cm²/ sec, or at least about 1cm²/ sec, or at least about 1.3cm²/ sec as measured according to Distribution Capacity Test.

Due to the patch having a fast acquisition time, the absorbent article of the present invention can instantly absorb the body fluid from the body, so that it cannot only effectively prevent fluid leaking but also provide a superior clean dry experience by limiting fluid spreading on the body.

Especially when the absorbent core comprises superabsorbent polymers which have a high absorption capacity but a relatively slow absorption speed, the patch of the present invention enables the absorbent core to fully utilize its high absorption capacity by distributing the fluid into a wide planar area and thus delivering the body fluid into a wide area of the absorbent core. However, as stated above, a high xy-planar distribution speed is advantageous in light of a planar direction of fluid distribution, however a colored area quickly reaches to the side edges of the absorbent article even with a small loading amount of body fluid which causes unnecessary consumers concern that the absorbent article will have a side leakage. Therefore, the patch needs to be designed sophisticatedly to balance distribution speeds and not cause unnecessary concern of side leakage due to the fast planar distribution speed.

According to the present invention, the patch has a width in the range of about 30% to about 80%, or about 35% to about 70%, or about 40% to about 6% of a width of the absorbent core along the transversal axis, and a length in the range of about 30% to about 75%, or about 35% to about 70%, or about 40% to about 60% of a length of the absorbent core along the longitudinal axis.

Exemplary materials suitable for the patch include a spacer woven fabric. A spacer woven fabric comprises a top face, a bottom face, and a plurality of yarns interconnecting the top and bottom faces, wherein the top and bottom faces are spaced apart from each other.

The top face and the bottom face of the spacer woven fabric may have the same configuration, or have different configurations in order to direct the fluid flow speed.

The spacer woven fabric may comprise thermoplastic fibers including polyesters, polyamides, polyolefins such as polyethylenes and polypropylenes, or any mixtures thereof. The spacer woven fabric may further comprise absorbent fibers. Optional absorbent fibers can provide absorption of liquid insults from the wearer-facing surface or topsheet. Any suitable absorbent material may be utilized for the absorbent fibers. Some examples of absorbent materials include cotton, pulp, rayon or regenerated cellulose, or combinations thereof. The spacer woven fabric may not comprise absorbent materials such as superabsorbent fibers and superabsorbent polymers. The top face and the bottom face of the spacer woven fabric may be made from the same fibers, or made from different fibers. At least one of the top face and the bottom face comprises polyethylene terephthalate (“PET”) fibers.

The spacer woven fabric may also contain surfactant to facilitate fluid penetration in order to be drained quickly and not hold fluid unnecessarily long, thus maintaining free volume capacity for the next gush of fluid.

The spacer woven fabric may have a basis weight from about 150 gsm to 350 gsm, or from about 200 gsm to about 300 gsm, or from about 150 gsm to about 250 gsm.

A caliper of the spacer woven fabric can be adjusted for optimizing a fluid acquisition speed and fit to the wearer's body. If the caliper is too small, it may negatively impact fluid acquisition speed. If the caliper is too high, an absorbent article comprising the spacer woven fabric may not fit well to the wearer's body.

Each of the yarns interconnecting the first and second faces in the spacer woven fabric disclosed herein may comprise about 2 filaments to about 60 filaments. Multiple filaments constituting yarns create inter-filament microchannels which enhance fluid wicking and faster fluid transport. Filaments constituting yarns may have a fineness of 1.5-10 dtex, or 3-8 dtex, or 2-5 dtex. If the filaments are too thin it may negatively impact on resilience of the spacer woven fabric and fluid acquisition speed. If the filaments are too thick, the material may be stiff with a higher rewet.

The top and bottom faces of the spacer woven fabric have may have openings in order to ensure a rapid inflow and effective distribution of body fluid. The bottom face of the spacer woven fabric may have a closer structure with relatively small openings than the top face. In one embodiment, the top face of the spacer woven fabric comprises openings, the openings have an opening area no less than about 0.2 mm² as measured according to Spacer Woven Fabric Dimension Test.

The yarn may form channels for fluid flow between the first and second faces in order to efficiently distribute and transfer the fluid to the absorbent core. In addition, due to the large void space between the first and second faces in the spacer woven fabric, the spacer woven fabric can accommodate and temporarily hold a relatively large fluid volume. Thus, body fluid discharged can be effectively received into the spacer woven fabric, and can flow it to an underlying component such as optional fluid management layer, or an absorbent core comprising an absorbent material where the body fluid is absorbed.

The spacer woven fabric is orientated in the absorbent article in such a way that the top face of the spacer woven fabric is facing the direction of the topsheet and the bottom face of the spacer woven fabric is facing the direction of the backsheet. In addition, due to the unique spacer woven fabric structure, the absorbent article of the present invention can have a high compression resistance without significant increase in a basis weight or volume of an absorbent article.

Especially when the absorbent core comprises superabsorbent polymers which have a high absorption capacity but a relatively slow absorption speed, the patch of the present invention enables the absorbent core to fully utilize its high absorption capacity by temporarily holding fluid volume and delivering the body fluid into a wider area of the absorbent core due to the material having a high planar distribution speed.

The patch is disposed between a topsheet and an absorbent core. The patch may be disposed between a topsheet and an optional fluid management layer. The patch may be disposed between an optional fluid management layer and an absorbent core.

Fluid management layer

The absorbent article of the invention may optionally comprise a fluid management layer 27 between the topsheet 24 and the absorbent core 28. The fluid management layer 27 can be disposed above or underneath the patch layer 25. The purpose of the fluid management layer is normally to readily transfer the acquired body fluid from the topsheet to the absorbent core, the transfer of fluid occurring not only vertically in the thickness of the fluid management layer, but also along the length and the width directions of the absorbent product. This helps the fluid capacity of the underlying storage layer to be fully utilized.

A fluid management layer may be manufactured from a wide range of materials such as woven, nonwoven materials, polymeric materials such as apertured formed thermoplastic films, apertured plastic film, hydro formed thermoplastic films, porous foams, reticulated foams, reticulated thermoplastic films and thermoplastic scrims. Any material described hereinbefore for the topsheet can be used for the fluid management layer.

In one embodiment, the fluid management layer completely covers an upper surface of the absorbent core. The fluid management layer may extend to the periphery of the topsheet so that the fluid management layer underlies the topsheet on the entire garment facing surface of the topsheet.

### Test Methods

### 1. Sample Preparation

If a patch is available in its raw material form, a specimen with the size about 25 mm×25 mm or a bigger size is cut from the raw material. If a patch is a component layer of an absorbent article, the absorbent article this size is cut and the patch is removed from the absorbent article, using a razor blade to excise the patch from the absorbent article. A cryogenic spray (such as Cyto-Freeze, Control Company, Houston TX) or other suitable solvents that do not permanently alter the properties of the patch composition may be used to remove the patch specimen from the other layers if necessary. Any remaining adhesive may be removed from the specimen by the following steps using tetrahydrofuran (THF) as solvent.
1) In a hood, transfer 1 liter of THF into the 3-4 liter beaker.
2) Submerge specimen in the 1 liter of THF.
3) Place beaker on shaking table and stir gently for 15 minutes and keep solution with sample sit for 5 additional minutes.
4) Take specimen out of THE solution, and carefully squeeze THE solution out of specimen.
5) Let specimen air dry in hood for a minimum of 15 minutes.

### 2. Absorbent Core and Patch Dimension Measurement

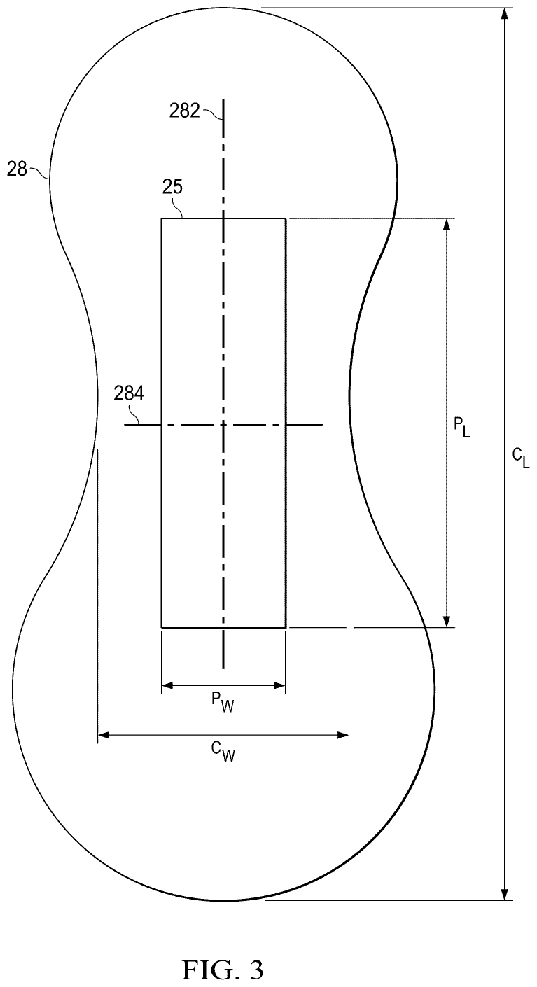

Referring to FIG. 3, a patch 25 has a patch longitudinal axis 282 and a patch transversal axis 284 perpendicular to the patch longitudinal axis 282. The longest length of the patch 25 parallel to the patch longitudinal axis 282 is determined as a patch length P_{L}. A width of the patch 25 on the patch transversal axis 284 is determined as a patch width P_{W}.

The longest length of the absorbent core 28 parallel to the patch longitudinal axis 282 is determined as an absorbent core length C_{L}. A width of the absorbent core 28 along the patch transversal axis 284 is determined as an absorbent core width C_{W}. That is, the absorbent core width C_{W} and the patch width P_{W} are on the same line.

### 3. Distribution Property Test

### 3.1 Material Acquisition Time Test

Material acquisition time of a material is measured according to WSP-70.3 Nonwoven Coverstock Liquid Strike-Through Time Using Simulated Urine.

### 3.2 XY-direction Distribution Test

0.9% saline with red dye is prepared. 10 μL saline is applied in a center of a test material via a pipette. After waiting for 90 s for absorption, acquire an image of the test material using a color scanner HP Scanjet G4050 or equivalent, and clean the scanner surface after each scan.

Image analysis is performed using image analysis program such as Image J software (version 1.52p or above, National Institute of Health, USA) or equivalent. The image needs to be distance calibrated with an image of a ruler to determine the image resolution.

Open the material image in Image J. Set the scale according to the image resolution. Crop the image in the center area to make a minimum bounding rectangular selection around the total stain region visible across multiple pad layers. Convert the image type to 8 bit. Apply a Gaussian blur filter to smooth the image by a Gaussian function with a Sigma (radius) of 2. The filtered 8-bit grayscale image is then converted to a binary image using the “Minimum” thresholding method to find the boundary of the stain region on the topsheet (as a result of fluid left on the topsheet) against the lighter-colored stain region from the subsequent layers.

The area of the selected stain region on the material is obtained and recorded as material stain size to the nearest 0.01 cm². This entire procedure is repeated on three substantially similar replicate articles. The average of the three individual recorded measurements to the nearest 0.01 cm² is reported as xy-direction distribution.

### 4. Acquisition Time Test

### 4.1 Artificial Menstrual Fluid (“AMF”) Preparation

AMF is composed of a mixture of defibrinated sheep blood, a phosphate buffered saline solution and a mucous component, and has a viscosity between 7.15 cSt to 8.65 cSt at 23±1° C.

Viscosity on the AMF is performed using a low viscosity rotary viscometer such as Cannon LV-2020 Rotary Viscometer with UL adapter (Cannon Instrument Co., State College, US) or equivalent. The appropriate size spindle for the viscosity range is selected, and instrument is operated and calibrated as per the manufacturer. Measurements are taken at 23° C. ±1° C. and at 60 rpm. Results are reported to the nearest 0.01 cSt.

### Defibrinated Sheep Blood

Defibrinated sheep blood with a packed cell volume of 38% or greater collected under sterile conditions (available from Cleveland Scientific, Inc., Bath, OH, US) or equivalent is used.

### Phosphate Buffered Saline Solution

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. Add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

### Mucous Component

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. A successful range of gastric mucin is usually between 38 to 50 grams. To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5° C. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range, then remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1° C.

Allow the temperature of the sheep blood and mucous component to come to 23° C.±1° C. Using a 500 mL graduated cylinder, measure the volume of the entire batch of the mucous component and add it to a 1200 ml beaker. Add an equal volume of sheep blood to the beaker and mix thoroughly. Using the viscosity method previously described, ensure the viscosity of the AMF is between 7.15-8.65 cSt. If not, the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1° C. Any unused portion is discarded after testing is complete.

### 4.2 Acquisition Time Measurement

Acquisition time is measured for an absorbent article loaded with AMF as described herein, using a strikethrough plate and an electronic circuit interval timer. The time required for the absorbent article to acquire a dose of AMF is recorded. All measurements are performed in a laboratory maintained at 23° C.±2° C. and 50% +2% relative humidity.

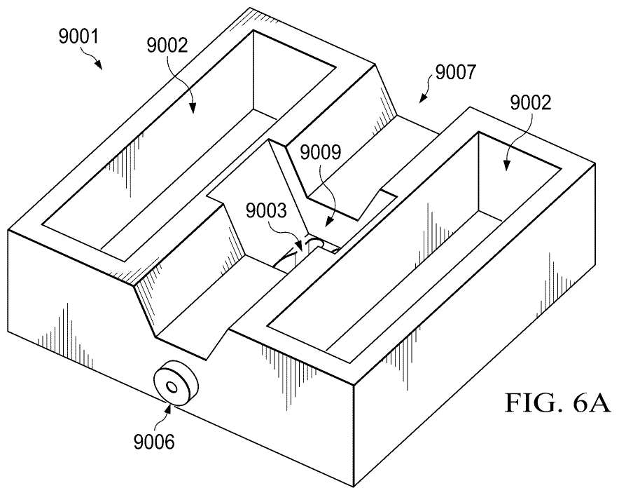

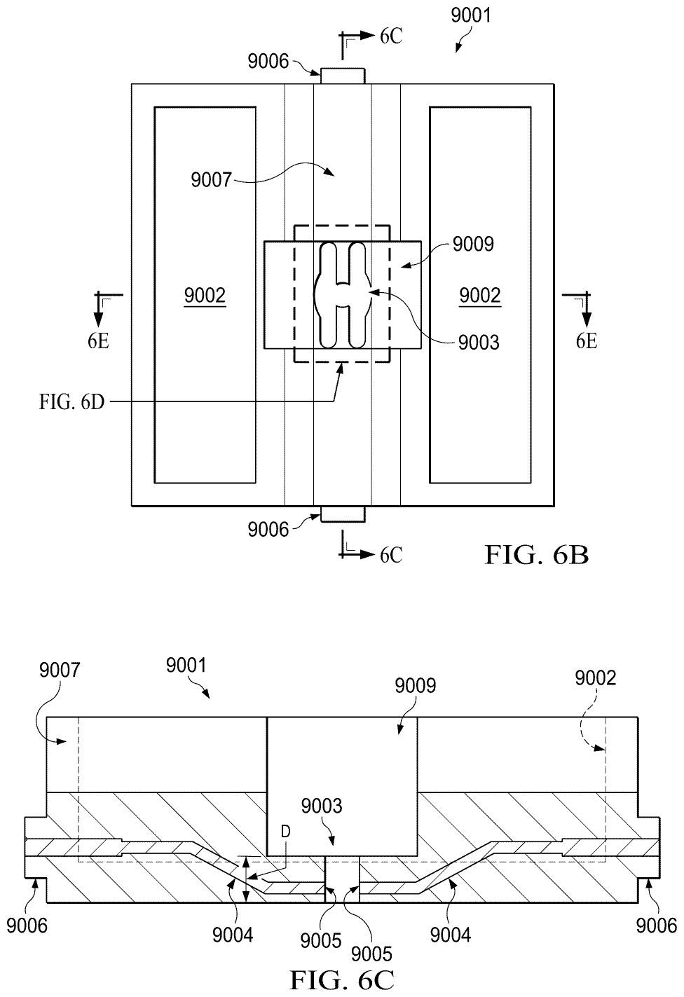

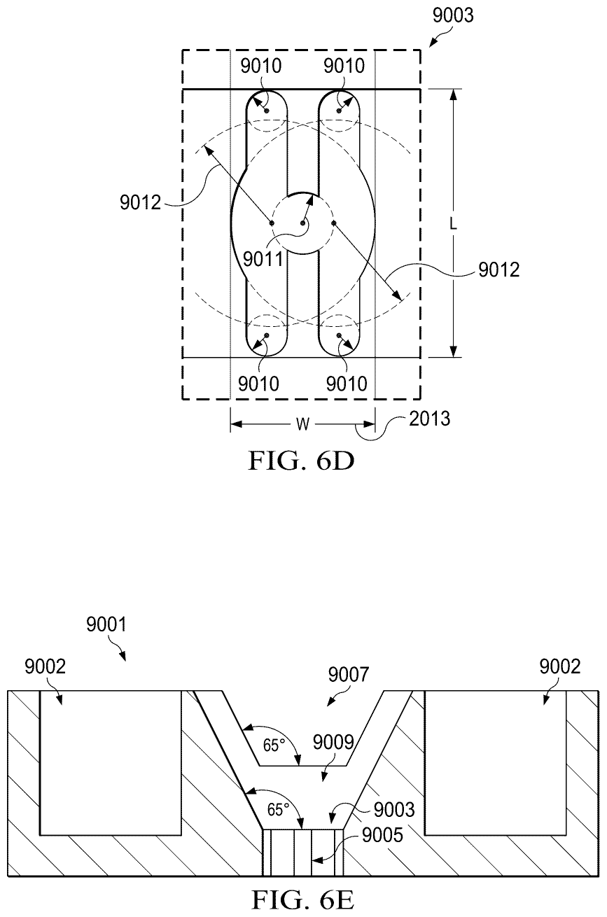

Referring to FIGS. 6A-6E, the strikethrough plate 9001 is constructed of Plexiglas with an overall dimension of 10.2 cm long by 10.2 cm wide by 3.2 cm tall. A longitudinal channel 9007 running the length of the plate is 13 mm deep and 28 mm wide at the top plane of the plate, with lateral walls that slope downward at 65° to a 15 mm wide base. A central test fluid well 9009 is 26 mm long, 24 mm deep and 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying article. The test fluid reservoir 9003 has an overall length (“L”) of 25 mm, width (“W”) of 15 mm, and depth (“D”) of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 9004 6 mm apart. The lateral sides of the reservoir bow outward at a radius 9012 of 14 mm bounded by the overall width, W, of 15 mm. Two wells 9002 (80.5 mm long×24.5 mm wide×25 mm deep) located outboard of the lateral channel, are filled with lead shot to adjust the overall mass of the plate to provide a constraining pressure of 0.25 psi (17.6 gf/cm²) to the test area. Electrodes 9004 are embedded in the plate 9001, connecting the exterior banana jacks 9006 to the inside wall of the fluid reservoir 9003. A circuit interval timer is plugged into the jacks 9006 to the inside wall 9005 of the fluid reservoir 9003. A circuit interval timer (not shown in the drawings) is plugged into the jacks 9006, and monitors the impedance between the two electrodes 9004, and measures the time from introduction of the AMF into reservoir 9003 until the AMF drains from the reservoir. The timer has a resolution of 0.01 sec.

Test products are removed from all packaging using care not to press down or pull on the products while handling. No attempt is made to smooth out wrinkles. The test samples are conditioned at 23° C.±2° C. and 50%±2% relative humidity for at least 2 hours prior to testing.

The required mass of the strikethrough plate must be calculated for the specific dimensions of the test article such that a confining pressure of 1.72 kPa is applied. Determine the longitudinal and lateral midpoint of the article's absorbent core. Measure and record the lateral width of the core to the nearest 0.1 cm. The required mass of the strikethrough plate is calculated as the core width multiplied by strikethrough plate length (10.2 cm) multiplied by 17.6 gf/cm² and recorded to the nearest 0.1 g. Add lead shot to the plate to achieve the calculated mass.

Connect the electronic circuit interval timer to the strikethrough plate 9001 and zero the timer. Place the test product onto a flat, horizontal surface with the body side facing up. Gently place the strikethrough plate 9001 onto the center of the test product ensuring that the “H” shaped reservoir 9003 is centered over the test area.

Using a mechanical pipette, accurately pipette 3.00 mL±0.05mL of AMF into the test fluid reservoir 9003. The fluid is dispensed, without splashing, along the molded lip of the bottom of the reservoir 9003 within a period of 3 seconds or less. After the fluid has been acquired, record the acquisition time to the nearest 0.01 second. Thoroughly clean the electrodes 9004 before each test.

In like fashion, a total of three replicate samples are tested for each test product to be evaluated. The arithmetic mean of the replicates is calculated to the nearest 0.01 sec, and reported as the Acquisition Time (sec).

### 5. Stain Size Test

A test absorbent article product is placed on a flat surface. 3 mL AMF is applied on a center of the test product via a pipette in 12 seconds. After 3 hours, quicky acquire an image of a topsheet of the test product using a color scanner HP Scanjet G4050 or equivalent, and clean the scanner surface after each scan. The image is analyzed to measure the stain size. Meanwhile, the stain width is measured using a ruler: draw a rectangular box inscribing around contour of the stain area ensuring that two sides of the rectangle are parallel to the core longitudinal axis 282 centerline of the pad (282). The distance between these two sides is determined as a stain width.

Image analysis is performed using image analysis program such as Image J software (version 1.52p or above, National Institute of Health, USA) or equivalent. The image needs to be distance calibrated with an image of a ruler to determine the image resolution.

Open a topsheet image in Image J. Set the scale according to the image resolution. Crop the image in the center area to make a minimum bounding rectangular selection around the total stain region visible across multiple pad layers. Convert the image type to 8 bit. Apply a Gaussian blur filter to smooth the image by a Gaussian function with a Sigma (radius) of 2. The filtered 8-bit grayscale image is then converted to a binary image using the “Minimum” thresholding method to find the boundary of the stain region on the topsheet (as a result of fluid left on the topsheet) against the lighter-colored stain region from the subsequent layers.

The area of the selected stain region on the topsheet is obtained and recorded as topsheet stain size to the nearest 0.01 cm². This entire procedure is repeated on three substantially similar replicate articles. The average of the three individual recorded measurements for a stain size to the nearest 0.01 cm² is reported to a stain size.

### EXAMPLES

### Example I. Patch Materials

Z-direction acquisition speed and xy-direction distribution power of various patch materials were tested according to the Distribution Property Test disclosed herein.
Patch material 1: KW-AC-15-20-N from Kingwonder, China. PET filaments 75D/36F (36 fibers, total thickness is 75 Denier.)
Patch material 2: Airlaid nonwoven, mixture of pulp and 2.2 dtex PE/PET bicomponent fiber.
Patch material 3: Spunlace nonwoven, mixture of 1.5 dtex PET fiber and 5.5 dtex PE/PET bicomponent fiber.
Patch material 4: Mixture of pulp and SAP, wrapped by tissue.
Patch material 5: Carded air-through nonwoven, 2.7 dtex PE/PET bicomponent fiber.

TABLE 1

| | Patch | Patch | Patch | Patch | Patch |
|---|---|---|---|---|---|
| | mate- | mate- | mate- | mate- | mate- |
| | rial 1 | rial 2 | rial 3 | rial 4 | rial 5 |
| Basis weight (gsm) | 215 | 195 | 90 | 170 | 65 |
| Material acquisition time (sec) | 2.45 | 5.73 | 5.86 | 14.20 | 3.61 |
| Xy-direction distribution | 1.96 | 0.62 | 0.59 | 0.59 | 0.12 |
| (cm²) | | | | | |

Patch material 1 has a very fast z-direction distribution speed and a xy-direction distribution compared to any of patch materials 2-5.

### Example II. Absorbent Article Preparation

Sanitary napkins as an exemplary absorbent article product having a patch material 1tested in Example 1 above in various patch sizes were prepared as indicated in Table 2-1. Sanitary napkins having a patch material 2 tested in Example 1 above in various patch sizes were prepared as indicated in Table 2-2. In each set of sanitary napkins, all sanitary napkins were prepared using a common topsheet, an absorbent core and a backsheet. The absorbent core is in a rectangle shape having a length of 216 mm and a width of 68 mm.

### Example III. Absorbent Article Test

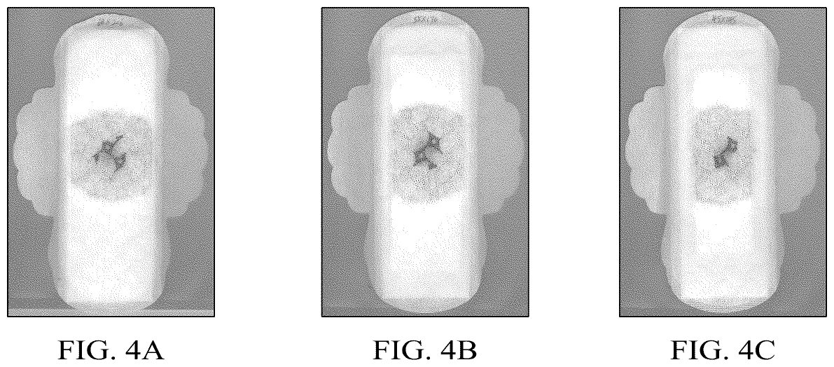

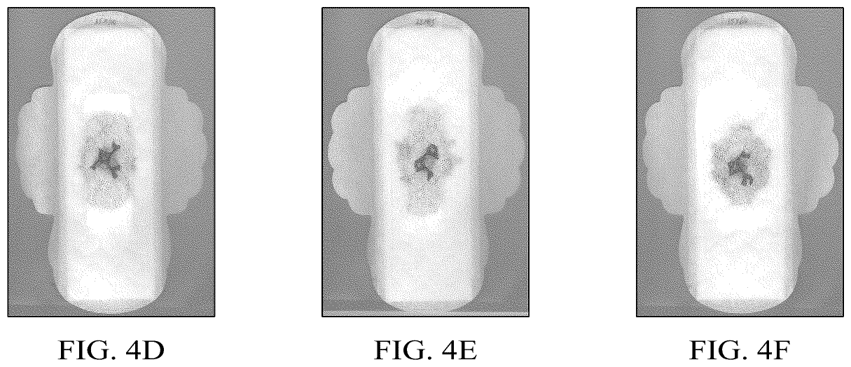

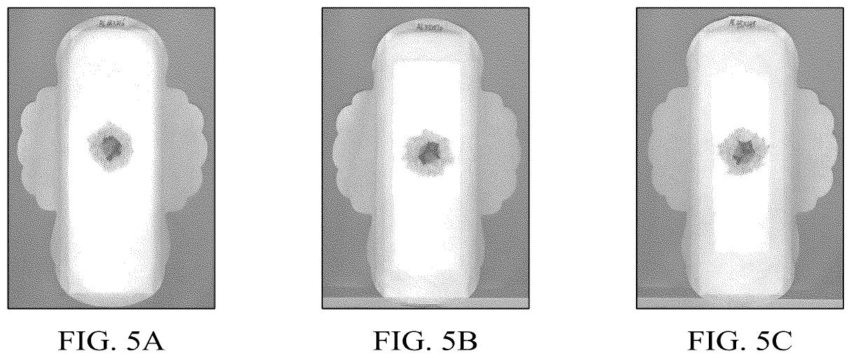

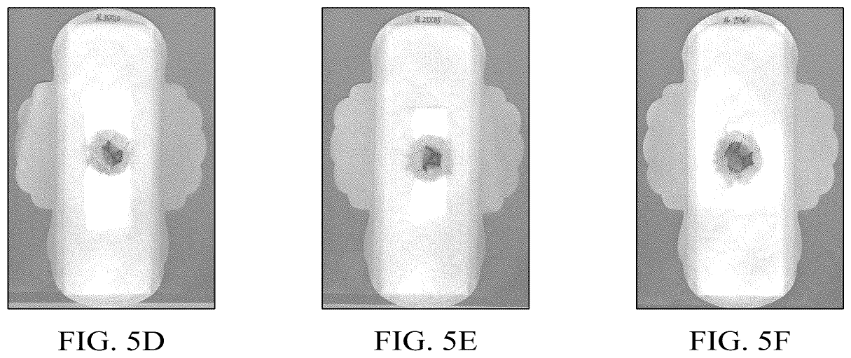

Acquisition time was evaluated on products prepared in Example II according to Acquisition Time Test under TEST METHODS, and results are indicated in Tables 2-1 and 2-2 below. Stain sizes were evaluated on products prepared in Example II according to Size Test under TEST METHODS. Images on a topsheet side of the products are shown in FIGS. 4A-4F for sanitary napkins having patches of patch material 1, and in FIGS. 5A-5F for sanitary napkins having patches of patch material 4. Stain sizes, stain lengths and stain widths are indicated in Tables 2-1 and 2-2 below.

TABLE 2-1

| | Product 1 | Product 2 | Product 3 | Product 4 | Product 5 | Product 6 |
|---|---|---|---|---|---|---|
| Patch width (mm) | 68 (100%) | 55 (81%) | 45 (66%) | 35 (51%) | 25 (37%) | 15 (22%) |
| Patch length | 216 (100%) | 170 (79%) | 145 (67%) | 110 (51%) | 85 (39%) | 60 (28%) |
| (mm) | | | | | | |
| Stain Images | FIG. 4A | FIG. 4B | FIG. 4C | FIG. 4D | FIG. 4E | FIG. 4F |
| Stain width (mm) | 66 | 60 | 53 | 49 | 53 | 50 |
| Stain size (cm²) | 47 | 47 | 40 | 34 | 32 | 26 |
| Acquisition Time | 2.5 | 2.7 | 2.7 | 2.6 | 2.8 | 3.5 |
| (sec) | | | | | | |

TABLE 2-2

| | Product 7 | Product 8 | Product 9 | Product 10 | Product 11 | Product 12 |
|---|---|---|---|---|---|---|
| Patch width (mm) | 68 (100%) | 55 (81%) | 45 (66%) | 35 (51%) | 25 (37%) | 15 (22%) |
| Patch length | 216(100%) | 170 (79%) | 145 (67%) | 110 (51%) | 85 (39%) | 60 (28%) |
| (mm) | | | | | | |
| Stain Images | FIG. 5A | FIG. 5B | FIG. 5C | FIG. 5D | FIG. 5E | FIG. 5F |
| Acquisition Time | 9.5 | None | None | None | None | None |
| (sec) | | | | | | |

As shown in FIGS. 4A-4F and confirmed in Table 2-1, Products 1-6, absorbent articles with patch material 1, have significantly fast acquisition time (sec) than Product 7, an absorbent articles with patch material 2.

Again, referring to FIGS. 4A-4F and Table 2-1, absorbent articles with patch material 1 vary in a stain size, a strain length and a stain width depending on a patch dimension and a patch size. When a patch has a width greater than about 80% of a width of the absorbent core, and a length greater than about 75% of a length of the absorbent core, the stain area reaches the side edges a product which may cause a side leakage issue and/or negatively impact on protection perception even if there is no side leakage.

Absorbent articles with patch material 2 have a quite similar size of stain area as shown FIGS. 5A-5F.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a liquid-permeable topsheet,
a backsheet,
an absorbent core disposed between the topsheet and the backsheet, and
a patch disposed between the topsheet and the absorbent core, the patch having a transversal axis,
wherein the patch has an acquisition time no higher than about 5 seconds as measured according to Material Acquisition Time Test,
wherein the patch has an xy-direction distribution of at least about 0.8 cm²/sec as measured according to XY-direction Distribution Test, and
wherein the patch has a width in the range of about 30% to about 80% of a width of the absorbent core, and a length in the range of about 30% to about 75% of a length of the absorbent core.

2. The absorbent article of claim 1, wherein the patch has an acquisition time no higher than about 3 seconds as measured according to Material Acquisition Time Test.

3. The absorbent article of claim 1, wherein the patch comprises spacer woven fabric comprising a top face, a bottom face, and a plurality of yarns interconnecting the first and second faces, wherein the first and second faces are spaced apart from each other.

4. The absorbent article of claim 1, wherein absorbent article comprises a secondary topsheet between the topsheet and the patch.

5. The absorbent article of claim 1, wherein the patch has a first color different from a second color of the topsheet.

6. The absorbent article of claim 1, wherein the absorbent article is a sanitary napkin or a menstrual pant.

7. The absorbent article of claim 1, wherein the absorbent article further comprises a pair of side flaps, the side flaps having a transversal axis.

8. The absorbent article of claim 7, wherein the transversal axis of the patch substantially match with the transversal axis of the pair of flaps.

9. An absorbent article comprising:
a liquid-permeable topsheet,
a backsheet,
an absorbent core disposed between the topsheet and the backsheet, and
a patch disposed between the topsheet and the absorbent core, the patch having a transversal axis,
wherein the patch has an acquisition time no higher than about 5 seconds as measured according to Material Acquisition Time Test,
wherein the patch has an xy-direction distribution of at least about 0.8cm²/sec as measured according to XY-direction Distribution Test,
wherein the patch has a width in the range of about 30% to about 80% of a width of the absorbent core, and a length in the range of about 30% to about 75% of a length of the absorbent core, and
wherein the patch comprises spacer woven fabric comprising a top face, a bottom face, and a plurality of yarns interconnecting the first and second faces, wherein the first and second faces are spaced apart from each other.

10. The absorbent article of claim 9, wherein absorbent article further comprises a secondary topsheet between the topsheet and the patch.

11. The absorbent article of claim 9, wherein the patch has a color different from a color of the topsheet.

12. The absorbent article of claim 9, wherein the spacer woven fabric comprises thermoplastic fibers, wherein the thermoplastic fibers may comprise at least one of polyesters, polyamides, and polyolefins.

13. The absorbent article of claim 12, wherein the spacer nonwoven fabric comprises polyolefins, and wherein the polyolefins is at least one of polyethylene and polypropylene.

14. The absorbent article of claim 9, wherein the spacer woven fabric has a basis weight from about 150 gsm to 350 gsm.

15. The absorbent article of claim 9, wherein the plurality of yarns have a fineness of from about 1.5 dtex to about 10 dtex.

16. The absorbent article of claim 9, wherein the plurality of yarns have a fineness of from about 3 dtex to about 8 dtex.

17. An absorbent article comprising:
a liquid-permeable topsheet,
a backsheet,
an absorbent core disposed between the topsheet and the backsheet, and
a patch disposed between the topsheet and the absorbent core, the patch having a transversal axis,
wherein the patch has an acquisition time no higher than about 5 seconds as measured according to Material Acquisition Time Test,
wherein the patch has an xy-direction distribution of at least about 0.8 cm²/sec as measured according to XY-direction Distribution Test,
wherein the patch has a width in the range of about 30% to about 80% of a width of the absorbent core, and a length in the range of about 30% to about 75% of a length of the absorbent core, and
wherein the patch comprises spacer woven fabric comprising a top face, a bottom face, and a plurality of yarns interconnecting the first and second faces, wherein the plurality of yarns have a fineness of from about 1.5 dtex to about 10 dtex.

18. The absorbent article of claim 17, wherein the spacer woven fabric has a basis weight from about 150 gsm to 350 gsm.

19. The absorbent article of claim 17, wherein absorbent article comprises a secondary topsheet between the topsheet and the patch.

20. The absorbent article of claim 17, wherein the patch has a first color and the topsheet has a second color, and wherein the first color and the second color are different.
